(19) United States
(12) Patent Application Publication
(10) Pub. No.: US 2010/0198338 A1
(43) Pub. Date: Aug. 5, 2010

(54) Hydrogen Sulfide Donating Polymers

(75) Inventors: Mingfei Chen, Santa Rosa, CA (US); Christopher Storment, Sonoma, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., A Delaware Corporation, Santa Rosa, CA (US)

(21) Appl. No.: 12/362,566

(22) Filed: Jan. 30, 2009

(51) Int. Cl. A61F 2/82; C08G 75/16; A61M 25/00

(52) U.S. Cl. 623/1.42; 528/364; 623/1.49; 604/523

(57) ABSTRACT

Described herein are hydrogen sulfide (H₂S) donating polymers and polymer systems suitable for coating or forming medical devices and methods for making and using the same. More specifically, described are H₂S donating polymers comprising at least one monomer with at least one basic group that can be complexed with H₂S to form a charged H₂S complex. The H₂S donating polymers can provide controlled release of H₂S once implanted at or within the target surgical site. The H₂S donating polymers can be coated onto a medical device, formed into a medical device or combined with one or more other polymers to form a polymer system.

### FIELD OF THE INVENTION

The present invention relates to hydrogen sulfide (H₂S) donating polymers for fabricating and coating medical devices.

### BACKGROUND OF THE INVENTION

For years, research in cardiovascular medicine has focused on the delivery of nitric oxide (NO) and carbon monoxide (CO), both of which are endogenously produced diatomic signaling molecules. It has been determined that therapies based on the administration of CO and NO protect the brain, heart and circulation against any number of cardiovascular diseases and conditions.

However, several studies have shown that both CO and NO treatments can be less than beneficial to a recipient. Although CO is beneficial for certain therapies, it has been known for decades to be a poisonous chemical in excess as it competes with carbon dioxide (CO₂) for preferential binding to hemoglobin in the blood. This preferential binding of CO leads to an excess of C0₂ in the blood and a detrimental state for the individual. NO, on the other hand, has been shown to be toxic at high concentrations due to the highly reactive nature of NO and its interaction with superoxide to form the potent oxidant peroxynitrite (ONOO⁻).

As a result of the two diatomic signaling molecule's acute toxicities, site specific administration has been pursued over the last decade, particularly of NO. Implantable medical devices such as vascular stents have been developed incorporating coatings which can provide controlled release of NO once implanted into a diseased vessel. This site specific administration of NO avoids the toxicity of a systemic administration, but does not avoid the effects of local ONOO⁻ formation.

Recently, a third endogenously produced signaling molecule, hydrogen sulfide (H₂S), has emerged as a candidate for cardiovascular therapy. Studies have shown that H₂S may be beneficial for vasodilatation, anti-inflammation and anti-restenosis. However, H₂S by nature is a toxic gas and, therefore, systemic administration is not a viable means for treating cardiovascular conditions. Therefore, methods of local administration of H₂S in order to utilize its vasodilating, anti-inflammation and anti-restenotic properties would be highly beneficial.

### SUMMARY OF THE INVENTION

Described herein are hydrogen sulfide (H₂S) donating polymers suitable for fabricating and coating medical devices and methods of making and using the same. More specifically, H₂S donating polymers are described comprising functional groups that can be reacted with H₂S to form a charged H₂S complex which releases or donates H₂S in a controlled manner.

Further described herein are hydrogen sulfide (H₂S) donating polymers comprising at least one basic group bound to H₂S. In one embodiment, the at least one basic group bound to H₂S comprises a polymer selected from the group consisting of polyesters, vinyl polymers, ether-ester polymers, polyanhydrides, phosphoester polymers, polyamines, polyamides, polyimines, polyimides, acrylic polymers, polycarbonates, polyolefins, polyurethanes, combinations and derivatives thereof.

Yet further described herein are polymers comprising: at least one monomer unit having a structure of Formula 1:

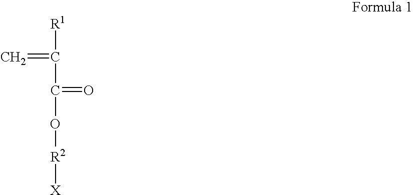

wherein R¹ is selected from hydrogen, C₁ to C₂₅ straight chain alkyl, C₃ to C₈ cyclic alkyl, C₂ to C₈ heterocycles, alkenyl groups, or poly alkenyl groups, or C₃ to C₂₅ branched alkyl, or any combination thereof; R² is selected from C₁ to C₂₅ straight chain alkyl, C₃ to C₈ cyclic alkyl, C₂ to C₈ heterocycles, alkenyl groups, or poly alkenyl groups, or C₃ to C₂₅ branched alkyl, or any combination thereof; and X is a charged H₂S complex having the structure of Formula 2

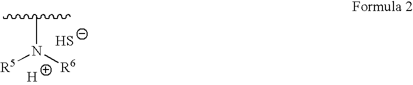

wherein R⁵ and R⁶ are each independently hydrogen or C₁ to C₂₅ straight chain alkyl.

In another embodiment, the H₂S donating polymer further comprises a second monomer selected from the group consisting of methyl methacrylate, butyl methacrylate, hexyl methacrylate, ethyl methacrylate, 2-(ethoxy ethylmethacrylate), methyl acrylate, ethyl acrylate, hexyl acrylate and butyl acrylate. In another embodiment, the H₂S donating polymer further comprises a third monomer selected from the group consisting of methyl methacrylate, butyl methacrylate, hexyl methacrylate, ethyl methacrylate, 2-(ethoxy ethylmethacrylate), methyl acrylate, ethyl acrylate, hexyl acrylate and butyl acrylate.

Further described herein are implantable medical devices comprising at least one basic group bound to H₂S. In one embodiment, the at least one basic group bound to H₂S comprises a polymer selected from the group consisting of polyesters, vinyl polymers, ether-ester polymers, polyanhydrides, phosphoester polymers, polyamines, polyamides, polyimines, polyimides, acrylic polymers, polycarbonates, polyolefins, polyurethanes, combinations and derivatives thereof.

In another embodiment, the at least one basic group bound to H₂S comprises: a H₂S donating polymer comprising the structure of Formula 4:

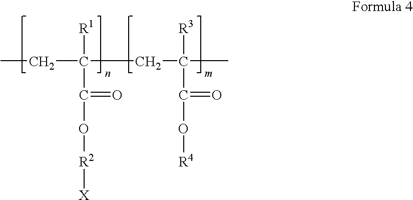

wherein R¹, R³ and R⁴ are each independently selected from hydrogen, C₁ to C₂₅ straight chain alkyl, C₃ to C₈ cyclic alkyl, C₂ to C₈ heterocycles, alkenyl groups, or poly alkenyl groups, or C₃ to C₂₅ branched alkyl, or any combination thereof; R² is selected from C₁ to C₂₅ straight chain alkyl, C₃ to C₈ cyclic alkyl, C₂ to C₈ heterocycles, alkenyl groups, or poly alkenyl groups, or C₃ to C₂₅ branched alkyl, or any combination thereof; n and m are each independently an integer between 1 and 25,000; and X is a charged H₂S complex having the structure of Formula 2:

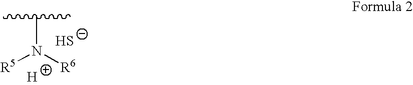

wherein R⁵ and R⁶ are each independently hydrogen, C₁ to C₂₅ straight chain alkyl.

In still another embodiment, the H₂S donating polymer of formula 4 further comprises one or more additional monomers selected from the group consisting of methyl methacrylate, butyl methacrylate, hexyl methacrylate, ethyl methacrylate, 2-(ethoxy ethylmethacrylate), methyl acrylate, ethyl acrylate, hexyl acrylate and butyl acrylate. In one embodiment, there exists a ratio of m to n and the ratio is between about 1:99 and about 99:1. In some embodiments, the ratio is between about 60:40 and about 40:60.

In one embodiment, described is an implantable medical device comprising a H₂S donating polymer of Formula 4

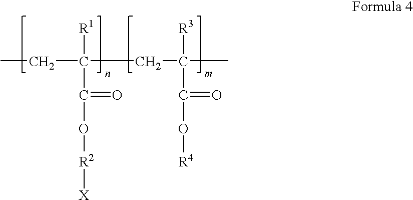

wherein R¹, R³ and R⁴ are each independently selected from hydrogen, C₁ to C₂₅ straight chain alkyl, C₃ to C₈ cyclic alkyl, C₂ to C₈ heterocycles, alkenyl groups, or poly alkenyl groups, or C₃ to C₂₅ branched alkyl, or any combination thereof; R² is selected from C₁ to C₂₅ straight chain alkyl, C₃ to C₈ cyclic alkyl, C₂ to C₈ heterocycles, alkenyl groups, or poly alkenyl groups, or C₃ to C₂₅ branched alkyl, or any combination thereof; n and m are each independently an integer between 1 and 25,000; and X is a charged H₂S complex having the structure of Formula 2:

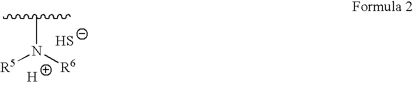

wherein R⁵ and R⁶ are each independently hydrogen, C₁ to C₂₅ straight chain alkyl.

In another embodiment, the H₂S donating polymer further comprises one or more additional monomers selected from the group consisting of methyl methacrylate, butyl methacrylate, hexyl methacrylate, ethyl methacrylate, 2-(ethoxy ethylmethacrylate), methyl acrylate, ethyl acrylate, hexyl acrylate and butyl acrylate. In one embodiment, there exists a ratio of m to n and the ratio is between about 1:99 and about 99:1. In one embodiment, the ratio is between about 60:40 and about 40:60.

In yet another embodiment, the implantable medical device is selected from the group consisting of stents, catheters, micro-particles, probes, vascular grafts, and combinations thereof. In another embodiment, the implantable medical device further comprises a parylene primer layer. In still another embodiment, the implantable medical device further comprises a cap coat.

In further embodiments, the H₂S donating polymer comprises one or more additional bioactive agents. In another embodiment, the one or more bioactive agent is selected from the group consisting of anti-proliferatives, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids. cytostatic compounds, toxic compounds, chemotherapeutic agents, analgesics, antibiotics, protease inhibitors, statins, nucleic acids, polypeptides, growth factors and delivery vectors, liposomes, and combinations thereof.

In one embodiment, described is a H₂S donating vascular stent comprising: a stent; and a polymer coating disposed upon the stent wherein the polymer has the composition of Formula 5

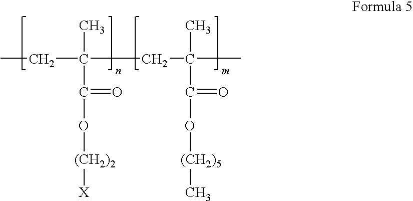

wherein X is a charged H₂S complex having the structure of Formula 2

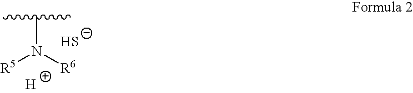

wherein R⁵ and R⁶ are each independently hydrogen, C₁ to C₂₅ straight chain alkyl.

In another embodiment, the stent further comprises a primer coating disposed on said stent. In yet another embodiment, the primer coat is parylene. In still another embodiment, the stent further comprises a cap coat disposed on the stent. In further embodiments, the cap coat is parylene.

### DEFINITION OF TERMS

Certain terms as used in the specification are intended to refer to the following definitions, as detailed below. Where the definition of terms departs from the commonly used meaning of the term, applicant intends to utilize the definitions provided below, unless specifically indicated.

Compatible: As used herein, “compatible” refers to a composition possessing the optimum, or near optimum combination of physical, chemical, biological and drug release kinetic properties suitable for a controlled-release coating made in accordance with the teachings of the present disclosure. Physical characteristics include durability and elasticity/ductility, chemical characteristics include solubility and/or miscibility and biological characteristics include biocompatibility. The drug release kinetic should be either near zero-order or a combination of first and zero-order kinetics.

Controlled release: As used herein “controlled release” refers to the release of a bioactive compound from a medical device surface at a predetermined rate. Controlled release implies that the bioactive compound does not come off the medical device surface sporadically in an unpredictable fashion and does not “burst” off of the device upon contact with a biological environment (also referred to herein a first order kinetics) unless specifically intended to do so. However, the term “controlled release” as used herein does not preclude a “burst phenomenon” associated with deployment. In some embodiments an initial burst of drug may be desirable followed by a more gradual release thereafter. The release rate may be steady state (commonly referred to as “timed release” or zero order kinetics), that is the drug is released in even amounts over a predetermined time (with or without an initial burst phase) or may be a gradient release. A gradient release implies that the concentration of drug released from the device surface changes over time.

Copolymer: As used herein, a “copolymer” will be defined as a macromolecule produced by the simultaneous chain addition polymerization of two or more dissimilar units such as monomers. Copolymer shall include bipolymers (two dissimilar units), terpolymers (three dissimilar units), etc.

Glass Transition Temperature (T_{g}): As used herein “glass transition temperature” or T_{g} refers to a temperature wherein a polymer structurally transitions from a elastic pliable state to a rigid and brittle state.

Mₙ: As used herein, Mₙ refers to number-average molecular weight. Mathematically it is represented by the following formula:

Mₙ=Σᵢ Nᵢ Mᵢ/Σᵢ Nᵢ, wherein the Nᵢ is the number of moles whose weight is Mᵢ.

M_{w}: As used herein, M_{w} refers to weight average molecular weight that is the average weight that a given polymer may have. Mathematically it is represented by the following formula:

M_{w}=Σᵢ Nᵢ Mᵢ²/Σᵢ Nᵢ Mᵢ, wherein Nᵢ is the number of molecules whose weight is Mᵢ.

### DETAILED DESCRIPTION OF THE INVENTION

Described herein are hydrogen sulfide (H₂S) donating polymers and polymer systems suitable for coating or forming medical devices and methods for making and using the same. More specifically, H₂S donating polymers are described comprising at least one basic group that can be complexed with H₂S. Suitable polymers that either include one or more basic groups that can be complexed with H₂S, or are suitable for the addition of one or more basic groups to the polymer as one or more pendant groups, include polyesters, vinyl polymers, ether-ester polymers, polyanhydrides, phosphoester polymers, polyamines, polyamides, polyimines, polyimides, acrylic polymers, polycarbonates, polyolefins, polyurethanes, and combinations and derivatives thereof.

Further, H₂S donating polymers, such as those listed above, are described comprising at least one monomer unit with at least one basic group that can be complexed with H₂S. Exemplary basic groups that can be complexed with H₂S include, but are not limited to, primary, secondary and tertiary straight chain amines, branched amines, cyclic amines, and straight and branched chain and cyclic carboxylates and phosphates. The basic groups can be reacted with H₂S to form a charged H₂S complex. The H₂S donating polymers provide controlled release of H₂S once implanted at or within the target site and can be coated onto a medical device, formed into a medical device or combined with one or more other polymers to form a polymer system suitable for the same.

In one embodiment, an exemplary H₂S donating polymer comprises at least one monomer unit of Formula 1.

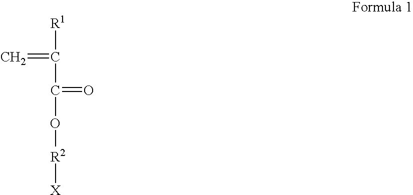

In Formula 1, R¹ is selected from hydrogen, C₁ to C₂₅ straight chain alkyl, C₃ to C₈ cyclic alkyl, C₂ to C₈ heterocycles, alkenyl groups, or poly alkenyl groups, or C₃ to C₂₅ branched alkyl, or any combination thereof and R² is selected from C₁ to C₂₅ straight chain alkyl, C₃ to C₈ cyclic alkyl, C₂ to C₈ heterocycles, alkenyl groups, or poly alkenyl groups, or C₃ to C₂₅ branched alkyl, or any combination thereof. X comprises one or more amine group that can form a charged H₂S complex which is depicted in Formula 2:

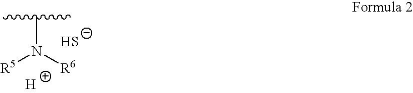

wherein R⁵ and R⁶ are each independently C₁ to C₁₀ straight chain alkyl. It is noted that X can be substituted at any position on R². If more than one X group is present, each X group can be located at a different position on R². In one embodiment, X is a charged H₂S complex having the structure of Formula 2.

In one embodiment, H₂S donating polymers can be formed comprising a monomer of Formula 1 and at least one additional monomer such as, but not limited to, methyl methacrylate, methyl butylmethacrylate, butyl methacrylate, hexyl methacrylate, ethyl acrylate, 2-(ethoxy ethylmethacrylate), methyl acrylate, ethyl acrylate, hexyl acrylate and butyl acrylate. In one embodiment, at least one of the above mentioned acrylic monomers comprises one or more tertiary amines capable of forming a charged H₂S complex. In another embodiment, monomers units of Formula 1 can be polymerized to form a homopolymer of Formula 3.

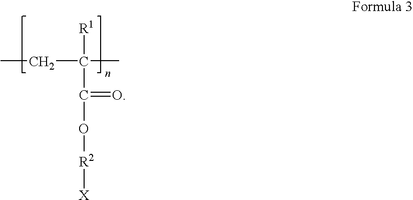

In Formula 3, R¹ and R² are as defined in Formula 1 above. Moreover, in Formula 3, n is an integer between about 2 and about 25,000. In other embodiments, n can be an integer between about 2 and about 5,000, about 2 and about 1,000, about 2 and about 500 or about 2 and about 100. X comprises a tertiary amine group that can form a charged H₂S complex as depicted in Formula 2 above.

In another embodiment, a polymer comprises a monomer unit of Formula 1 plus at least one additional monomer. An exemplary copolymer comprising a monomer unit of Formula 1 and an additional acrylate monomer is depicted in Formula 4.

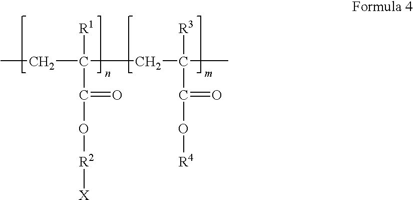

In Formula 4, R¹, R³, and R⁴ are each independently selected from hydrogen, C₁ to C₂₅ straight chain alkyl, C₃ to C₈ cyclic alkyl, C₂ to C₈ heterocycles, alkenyl groups, or poly alkenyl groups, or C₃ to C₂₅ branched alkyl, or any combination thereof, and R² is selected from C₁ to C₂₅ straight chain alkyl, C₃ to C₈ cyclic alkyl, C₂ to C₈ heterocycles, alkenyl groups, or poly alkenyl groups, or C₃ to C₂₅ branched alkyl, or any combination thereof. Further, in Formula 4, n and m are each independently an integer between about 1 and about 25,000 and b is an integer between 0 and 20. In other embodiments, n and m can each independently be an integer between about 1 and about 5,000, about 1 and about 1,000, about 1 and about 500 or about 1 and about 100. The sum of m and n is at least 2. X comprises one or more tertiary amine groups that can form a charged H₂S complex as depicted in Formula 2 above. In one embodiment, X is a charged H₂S complex having the structure of Formula 2.

With regard to Formula 4, the ratio of m to n (m:n) is between about 1:100 and about 100:1. In some embodiments, the ratio of m to n is 1:99, 10:90, 20:80, 30:70, 40:60, 50:50; 60:40, 70:30, 80:20, 90:10 and 99:1.

In an exemplary embodiment, a H₂S donating polymer comprises the structure of Formula 4 and substituents wherein R₁ is methyl, R₂ is ethyl, R₃ is methyl, R₄ is n-hexyl and X is a tertiary amine group that can form a charged H₂S complex as described above. In one embodiment, X is a charged H₂S complex having the structure of Formula 2. In another embodiment, the ratio of m to n ranges from about 1:100 to about 100:1 or m to n is about 73:27. In other embodiments, the ratio of m to n is about 43:57, more preferably is about 19:81. In other embodiments, the ratio of m to n is about 15:85, about 25:75, about 35:65, about 45:55, about 55:45, about 65:35, about 75:25 or about 85:15. In a further embodiment, the ratio of m to n is about 1:99 to about 99:1. The structure of such a H₂S donating polymer is depicted by Formula 5.

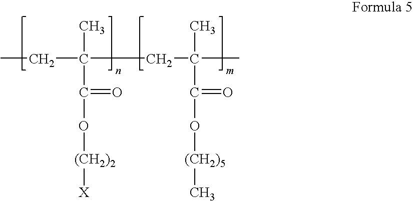

In another embodiment, the H₂S donating polymer of Formula 4 can further comprise one or more additional monomers. A terpolymer according to the present disclosure, for example, can include Formula 6.

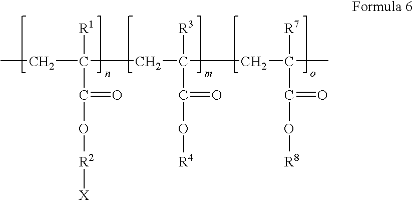

In Formula 5, R¹, R³, R⁴, R⁷ and R⁸ are each independently selected from hydrogen, C₁ to C₂₅ straight chain alkyl, C₃ to C₈ cyclic alkyl, C₂ to C₈ heterocycles, alkenyl groups, or poly alkenyl groups, or C₃ to C₂₅ branched alkyl, or any combination thereof, and R² is selected from C₁ to C₂₅ straight chain alkyl, C₃ to C₈ cyclic alkyl, C₂ to C₈ heterocycles, alkenyl groups, or poly alkenyl groups, or C₃ to C₂₅ branched alkyl, or any combination thereof. Further, in Formula 5, n, m and o are each independently an integer between about 1 and about 25,000. In other embodiments, n, m and o can each independently be an integer between about 1 and about 5,000, about 1 and about 1,000, about 1 and about 500 or about 1 and about 100. The sum of m, n and o is at least 3, and n must be at least 1. X comprises one or more tertiary amine groups that can form a charged H₂S complex as depicted in Formula 2 above. In one embodiment, X is a charged H₂S complex having the structure of Formula 2.

In one embodiment, the H₂S donating polymers and polymeric coatings described herein have one or more amine groups that can form charged H₂S complexes and upon exposure to a physiological medium can achieve controlled release of H₂S. In one embodiment, the amine group is a tertiary amine. Without wishing to be bound by theory, it is believed that a tertiary amine group, when subjected to a H₂S source, for example a gas, H₂S reacts with the tertiary amine thereby forming a charged H₂S complex. It is thought that H₂S will destabilize the tertiary amine group enough to allow formation of the charged H₂S complex. The reaction is depicted below (Scheme 1).

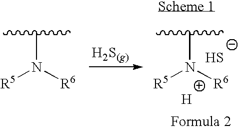

Physical properties of the H₂S donating polymers described herein can be fine tuned to optimally perform for their intended use. Properties that can be fine tuned, without limitation, include T_{g}, molecular weight (both Mₙ and M_{w}), polydispersity index (PDI, the quotient of M_{w}/Mₙ), degree of elasticity and degree of amphiphlicity. In one embodiment, the T_{g} of the polymers range from about −10° C. to about 85° C. In still another embodiment, the PDI of the polymers range from about 1.35 to about 4. In another embodiment, the T_{g} of the polymers ranges form about 0° C. to about 40° C. In still another embodiment, the PDI of the polymers range from about 1.5 to about 2.5.

In an exemplary embodiment, the H₂S donating polymeric coatings described herein are used to coat medical devices deployed in a hemodynamic environment. As such, in some embodiments, the H₂S donating polymers possess excellent adhesive properties. That is, the coating has the ability to be stably coated on the medical device surface.

The medical devices used may be permanent medical implants, temporary implants, or removable devices. For example, and not intended as a limitation, the medical devices may include stents, catheters, micro-particles, probes, and vascular grafts.

In one embodiment, the medical device is a stent or stents. The stents may be vascular stents, urethral stents, biliary stents, or stents intended for use in other ducts and organ lumens. Vascular stents, for example, may be used in peripheral, cerebral, or coronary applications. The stents may be rigid expandable stents or pliable self-expanding stents. Many different materials can be used to fabricate the implantable medical devices including, but not limited to, stainless steel, nitinol, aluminum, chromium, titanium, gold, cobalt, ceramics, and a wide range of synthetic polymeric and natural materials including, but not limited to, collagen, fibrin and plant fibers. All of these materials, and others, may be used with the polymeric coatings made in accordance with the teachings disclosed herein. Furthermore, the H₂S donating polymers described herein can be used to fabricate an entire medical device.

The stents may also be bioresorbable. In one embodiment, vascular stents are implanted into coronary arteries immediately following angioplasty. In another embodiment, vascular stents are implanted into the abdominal aorta to treat an abdominal aneurysm.

In another embodiment, the H₂S donating polymeric coatings are non-bioresorbable or substantially non-bioresorbable. A “non-bioresorbable” H₂S donating polymeric coating as used herein is biocompatible and not subject to breakdown in vivo through the action of normal biochemical pathways. In one embodiment, the H₂S donating polymeric coatings are substantially non-bioresorbable and remain greater than 95% intact after 1 year of implantation. In other embodiments, the substantially non-bioresorbable H₂S donating polymeric coatings remain greater than 90% intact after 1 year.

In another embodiment, the H₂S donating polymeric coatings are bioresorbable, meaning the H₂S donating polymeric coatings are biocompatible and are broken down in vivo through the action of normal biochemical pathways. In one embodiment, the H₂S donating polymeric coatings are bioresorbable and remain less than 5% intact after 1 year of implantation. In other embodiments, the H₂S donating polymeric coatings are bioresorbable and remain less than 5% intact after 2 years of implantation. In other embodiments, the H₂S donating polymeric coatings are bioresorbable and remain less than 5% intact after 5 years of implantation.

The H₂S donating polymers and associated polymeric coatings described herein can be formed as linear or branched polymers. Additionally, the polymers themselves can be formed as thermosets in order to attain a specific shape.

Further, the H₂S donating polymers and associated polymeric coatings described herein can be formed as a copolymer with one or more other monomers. The copolymer can be randomly assembled or can be a block copolymer wherein the polymer is formed with blocks of various monomers. One skilled in the art understands that copolymers can be fine tuned depending on, for example, monomer ratios, number of different monomers used (e.g. biopolymer, terpolymer), monomer hydrophobicity or hydrophilicity, monomer molecular weight, polymer molecular weight, catalyst used and polymerization temperature.

There are many theories that attempt to explain, or contribute to our understanding of how polymers adhere to surfaces. The most important forces include electrostatic and hydrogen bonding. However, other factors including wettability, absorption and resiliency also determine how well a polymer will adhere to different surfaces. Therefore, polymer base coats, or primers are often used in order to create a more uniform coating surface.

The H₂S donating polymeric coatings described herein can be applied to medical device surfaces, either primed or bare, in any manner known to those skilled in the art. Application methods for the H₂S donating polymeric coatings include, but are not limited to, spraying, dipping, brushing, vacuum-deposition, and the like. Moreover, in some embodiments, the H₂S donating polymeric coatings may be used with a cap coat. A cap coat as used herein refers to the outermost coating layer applied over another coating.

In one embodiment, a primer coating is applied to the surface of a stent or other implantable medical device. Then a H₂S donating polymer coating is applied over the primer coat. Thereafter, a polymer cap coat can be applied over the H₂S donating polymeric coating. The cap coat may optionally serve as a diffusion barrier to control the H₂S release. The cap coat may be merely a biocompatible polymer applied to the surface of the sent to protect the stent and have no effect on the H₂S release rates.

One or more additional polymer coatings may be applied to the medical device in any position relative to the medical device surface. For example, the additional layer may be between the primer layer and the H₂S donating layer or may be between the H₂S donating layer and the cap coat. Further, the additional layer may be on top of the cap coat.

The additional coating may further comprise one or more additional bioactive agents. The bioactive agent may further be incorporated into the H₂S donating layer. The choice of bioactive agent to incorporate, or how much to incorporate, will have a great deal to do with the polymer selected to coat or form the implantable medical device. A person skilled in the art will appreciate that hydrophobic agents prefer hydrophobic polymers and hydrophilic agents prefer hydrophilic polymers. Therefore, coatings and medical devices can be designed for agent or agent combinations with immediate release, sustained release or a combination of the two.

Exemplary, non limiting examples of bioactive agents that can be incorporated into the polymers and polymeric coating presently described include anti-proliferatives including, but not limited to, macrolide antibiotics including FKBP-12 binding compounds, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids. Drugs can also refer to bioactive agents including anti-proliferative compounds, cytostatic compounds, toxic compounds, anti-inflammatory compounds, chemotherapeutic agents, analgesics, antibiotics, protease inhibitors, statins, nucleic acids, polypeptides, growth factors and delivery vectors including recombinant micro-organisms, liposomes, and the like.

Exemplary FKBP-12 binding agents include sirolimus (rapamycin), tacrolimus (FK506), everolimus (certican or RAD-001), temsirolimus (CCI-779 or amorphous rapamycin 42-ester with 3-hydroxy-2-(hydroxymethyl)-2-methylpropionic acid as disclosed in U.S. patent application Ser. No. 10/930,487) and zotarolimus (ABT-578; see U.S. Pat. Nos. 6,015,815 and 6,329,386). Additionally, other rapamycin hydroxyesters as disclosed in U.S. Pat. No. 5,362,718 may be used in combination with the polymers described herein.

In one embodiment, the polymer chosen for a primer layer or as cap coats is preferably a polymer that is biocompatible and minimizes irritation to the vessel wall when the medical device is implanted. The polymer may be either a biostable, bioabsorbable or bioresorbable polymer depending on the desired rate, when used as a cap coat, of release or the desired degree of polymer stability. Bioabsorbable polymers that can be used include poly(L-lactic acid), polycaprolactone, poly(lactide-co-glycolide), poly(ethylene-vinyl acetate), poly(hydroxybutyrate-co-val erate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g. PEO/PLA), polyalkylene oxalates, polyphosphazenes and biomolecules such as fibrin, fibrinogen, cellulose, starch, collagen and hyaluronic acid.

Also, biostable polymers with a relatively low chronic tissue response such as polyurethanes, silicones, and polyesters could be used and other polymers could also be used if they can be dissolved and cured or polymerized on the medical device such as polyolefins, polyisobutylene and ethylene-alphaolefin copolymers; acrylic polymers and copolymers, ethylene-co-vinylacetate, polybutylmethacrylate, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins, polyurethanes; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose.

In an exemplary embodiment, the primer coat is parylene applied to a metal stent. Parylene can provide scaffolding on the medical device for other polymers or polymer systems. In such an embodiment, the H₂S donating polymer can be directly applied to the primer layer or to one or more layers applied to the primer layer.

Although it is within the scope of the present disclosure that additional bioactive agents can be useful in treating a plethora of medical conditions, in some exemplary embodiments, the use of a H₂S donating polymer can alleviate the need for additional bioactive agents. The H₂S donating polymers described herein have the effect of providing cardiovascular effects such as, but not limited to, vasodilatation, anti-inflammation and anti-restenosis. Therefore, medical devices incorporating H₂S donating polymers or polymer systems can have the benefit of alleviating the need for supplemental bioactive agents to treat vasoconstriction, inflammation and restenosis. Removing such bioactive agents from a patient's post implantation treatment can help reduce side effects associated with the systemic, or even local, administration of such agents.

Additionally, removing such agents from systemic administration or local delivery from the same medical device can reduce the complexity of the treatment. For example, some bioactive agents may not work well together or may require separate polymer systems in order to achieve controlled release from the implanted device.

### EXAMPLES

The following Examples are intended to illustrate a non-limiting process for coating metallic stents with a H₂S donating polymeric coating. One non-limiting example of a suitable metallic stent is the Medtronic/AVE S670™ 316L stainless steel coronary stent.

### Example 1

### Metal Stent Cleaning Procedure

Stainless steel stents are placed a glass beaker and covered with reagent grade or better hexane. The beaker containing the hexane immersed stents is then placed into an ultrasonic water bath and treated for 15 min at a frequency of between approximately 25 to 50 KHz. Next the stents are removed from the hexane and the hexane is discarded. The stents are then immersed in reagent grade or better 2-propanol and vessel containing the stents and the 2-propanol is treated in an ultrasonic water bath as before. Following cleaning the stents with organic solvents, they are thoroughly washed with distilled water and thereafter immersed in 1.0 N sodium hydroxide (NaOH) solution and treated at in an ultrasonic water bath as before. Finally, the stents are removed from the NaOH, thoroughly rinsed in distilled water and then dried in a vacuum oven overnight at 40° C. After cooling the dried stents to room temperature in a desiccated environment, they are weighed and their weights recorded.

### Example 2

### Synthesizing a Tertiary Amine Containing Polymer

Synthesis of hexyl methacrylate (HMA) and 2-dimethylaminoethyl methacrylate (DMEMA) copolymer is accomplished according to scheme 2:

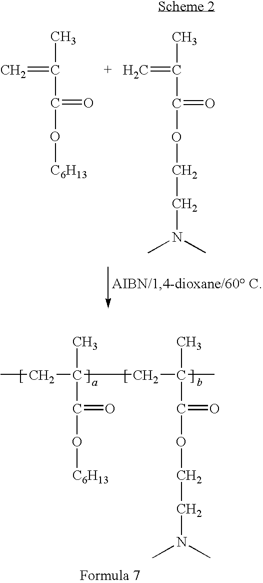

A glass bottle with a magnetic spin bar was charged with 80 mg of 2,2′-azodiisobutyronitrile (AIBN), 20.0 g of 1,4-dioxane, dimethylaminoethyl methacrylate and n-hexyl methacrylate according to Table 1. The bottle was sealed with a septum and purged with nitrogen for 30 min. The bottle was heated in an oil bath for 3 hr with stirring. Polymers were purified by precipitating the in methanol or hexanes. The isolated polymers were dried in an oven under high vacuum at 45° C. overnight. The polymers were characterized by nuclear magnetic resonance spectroscopy (NMR), Gel permeation chromatography (GPC) and differential scanning calorimetry (DSC). The properties of the polymers are listed in Table 2.

TABLE 1
Polymerization Formulation

| | | | | 1,4- |
|---|---|---|---|---|
| Formulation Code | AIBN (mg) | HMA (g) | DMAEMA (g) | dioxane(g) |
| 1948_040_#1 | 80 | 9.5 | 0.5 | 20 |
| 1948_040_#2 | 80 | 9 | 1 | 20 |
| 1948_040_#3 | 80 | 8 | 2 | 20 |
| 1948_040_#4 | 80 | 7 | 3 | 20 |
| 1948_040_#5 | 80 | 6 | 4 | 20 |

TABLE 2
Polymer Properties

| Polymer Code | DMAEMA (mol %) | M_{w} | PDI | T_{g} (° C.) |
|---|---|---|---|---|
| 1948_040_#1 | 5.8 | 157000 | 1.79 | −9.3 |
| 1948_040_#2 | 10.7 | 138000 | 1.8 | −5.1 |
| 1948_040_#3 | 19.9 | 122000 | 1.76 | −3.4 |
| 1948_040_#4 | 32.6 | 159000 | 1.52 | −3.1 |
| 1948_040_#5 | 43.0 | 159000 | 2.00 | −2.3 |

### Example 3

### Coating a Cleans Dried Metal Stent with a Tertiary Amine Containing Polymer

A tertiary amine containing polymer (Formula 7 from Example 2), can be coated onto a metal stent by dipping the clean dried metal stent from Example 1 into tetrahydrofuran (THF) solution of the tertiary amine containing polymer. In alternate examples, the polymeric coating can be applied by brushing or spraying the clean dried metal stent.

### Example 4

### The Formation of Stents Coated with a Charged H₂S Complex Using a Dry Environment and a H₂S Gas Source

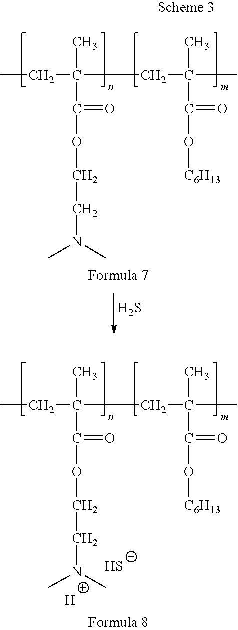

A vascular stent coated with a polymer of Formula 7 from Example 2 is placed in a 250 mL stainless steel PARR® (Parr Instrument Co., IL) mixing apparatus. The apparatus is degassed by repeated cycles (×10) of pressurization/depressurization with nitrogen gas at 10 atmospheres. Next, the vessel undergoes two cycles of pressurization/depressurization with H₂S at 30 atmospheres. Finally, the vessel is filled with H₂S at 30 atmospheres and left at room temperature for 24 hr. After 24 hr, the vessel is purged of H₂S and pressurized/depressurized with repeated cycles (×10) of nitrogen gas at 10 atmospheres. The stent is removed from the apparatus. This procedure results in a vascular stent coating with a polymer containing charged H₂S complexes (Scheme 3).

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

Specific embodiments disclosed herein may be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

What is claimed is:

1. A hydrogen sulfide (H₂S) donating polymer comprising a polymer having at least one basic group bound to H₂S.

2. The H₂S donating polymer of claim 1 wherein said polymer is selected from the group consisting of polyesters, vinyl polymers, ether-ester polymers, polyanhydrides, phosphoester polymers, polyamines, polyamides, polyimines, polyimides, acrylic polymers, polycarbonates, polyolefins, polyurethanes, combinations and derivatives thereof.

3. The hydrogen sulfide donating polymer of claim 1 wherein the basic group is selected from the group consisting of primary, secondary and tertiary straight chain amines, branched amines, cyclic amines, and straight and branched chain and cyclic carboxylates and phosphates

4. The H₂S donating polymer of claim 1 wherein said polymer comprises:
at least one monomer unit having a structure of Formula 1: 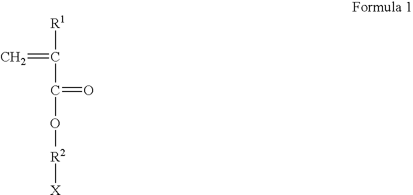
wherein R¹ is selected from hydrogen, C₁ to C₂₅ straight chain alkyl, C₃ to C₈ cyclic alkyl, C₂ to C₈ heterocycles, alkenyl groups, or poly alkenyl groups, or C₃ to C₂₅ branched alkyl, or any combination thereof;
R² is selected from C₁ to C₂₅ straight chain alkyl, C₃ to C₈ cyclic alkyl, C₂ to C₈ heterocycles, alkenyl groups, or poly alkenyl groups, or C₃ to C₂₅ branched alkyl, or any combination thereof; and
X is a charged H₂S complex having the structure of Formula 2 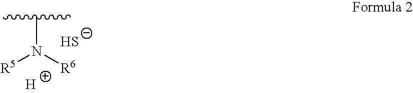
wherein R⁵ and R⁶ are each independently hydrogen or C₁ to C₂₅ straight chain alkyl.

5. The H₂S donating polymer of claim 4 further comprising a second monomer selected from the group consisting of methyl methacrylate, butyl methacrylate, hexyl methacrylate, ethyl methacrylate, 2-(ethoxy ethylmethacrylate), methyl acrylate, ethyl acrylate, hexyl acrylate and butyl acrylate.

6. The H₂S donating polymer of claim 5 further comprising a third monomer selected from the group consisting of methyl methacrylate, butyl methacrylate, hexyl methacrylate, ethyl methacrylate, 2-(ethoxy ethylmethacrylate), methyl acrylate, ethyl acrylate, hexyl acrylate and butyl acrylate.

7. A H₂S donating polymer comprising the structure of Formula 4: 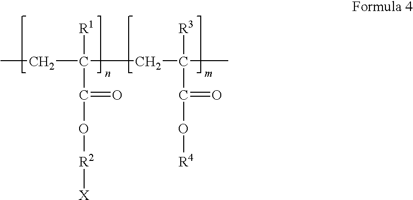
wherein R¹, R³ and R⁴ are each independently selected from hydrogen, C₁ to C₂₅ straight chain alkyl, C₃ to C₈ cyclic alkyl, C₂ to C₈ heterocycles, alkenyl groups, or poly alkenyl groups, or C₃ to C₂₅ branched alkyl, or any combination thereof;
R² is selected from hydrogen, C₁ to C₂₅ straight chain alkyl, C₃ to C₈ cyclic alkyl, C₂ to C₈ heterocycles, alkenyl groups, or poly alkenyl groups, or C₃ to C₂₅ branched alkyl, or any combination thereof;
n and m are each independently an integer between 1 and 25,000; and
X is a charged H₂S complex having the structure of Formula 2: 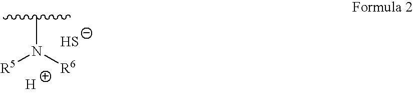
wherein R⁵ and R⁶ are each independently hydrogen or C₁ to C₂₅ straight chain alkyl.

8. The H₂S donating polymer of claim 7 further comprising one or more additional monomers selected from the group consisting of methyl methacrylate, butyl methacrylate, hexyl methacrylate, ethyl methacrylate, 2-(ethoxy ethylmethacrylate), methyl acrylate, ethyl acrylate, hexyl acrylate and butyl acrylate.

9. The H₂S donating polymer of claim 7 wherein there exists a ratio of m to n and said ratio is between about 1:99 and about 99:1.

10. The H₂S donating polymer of claim 9 wherein said ratio is between about 60:40 and about 40:60.

11. An implantable medical device comprising a polymer having at least one basic group bound to H₂S.

12. The implantable medical device of claim 11 wherein said polymer is selected from the group consisting of polyesters, vinyl polymers, ether-ester polymers, polyanhydrides, phosphoester polymers, polyamines, polyamides, polyimines, polyimides, acrylic polymers, polycarbonates, polyolefins, polyurethanes, combinations and derivatives thereof.

13. The implantable medical device of claim 11 wherein the basic group is selected from the group consisting of primary, secondary and tertiary straight chain amines, branched amines, cyclic amines, and straight and branched chain and cyclic carboxylates and phosphates

14. The implantable medical device of claim 11 wherein said polymer comprises:
a H₂S donating polymer of Formula 4 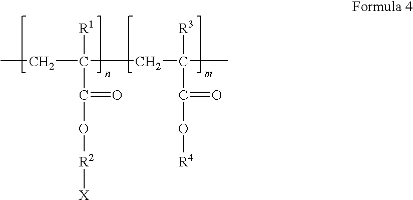
wherein R¹, R³ and R⁴ are each independently selected from hydrogen, C₁ to C₂₅ straight chain alkyl, C₃ to C₈ cyclic alkyl, C₂ to C₈ heterocycles, alkenyl groups, or poly alkenyl groups, or C₃ to C₂₅ branched alkyl, or any combination thereof;
R² is selected from C₁ to C₂₅ straight chain alkyl, C₃ to C₈ cyclic alkyl, C₂ to C₈ heterocycles, alkenyl groups, or poly alkenyl groups, or C₃ to C₂₅ branched alkyl, or any combination thereof;
n and m are each independently an integer between 1 and 25,000; and
X is a charged H₂S complex having the structure of Formula 2: 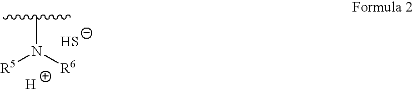
wherein R⁵ and R⁶ are each independently hydrogen or C₁ to C₂₅ straight chain alkyl.

15. The implantable medical device of claim 14 further comprising one or more additional monomers selected from the group consisting of methyl methacrylate, butyl methacrylate, hexyl methacrylate, ethyl methacrylate, 2-(ethoxy ethylmethacrylate), methyl acrylate, ethyl acrylate, hexyl acrylate and butyl acrylate.

16. The implantable medical device of claim 14 wherein there exists a ratio of m to n and said ratio is between about 1:99 and about 99:1.

17. The implantable medical device of claim 14 wherein said ratio is between about 60:40 and about 40:60.

18. The implantable medical device of claim 11 wherein said implantable medical device is selected from the group consisting of stents, catheters, micro-particles, probes, vascular grafts, and combinations thereof.

19. The implantable medical device of claim 14 further comprising a parylene primer layer.

20. The implantable medical device of claim 14 further comprising a cap coat.

21. The implantable medical device of claim 11 wherein said H₂S donating polymer comprises one or more additional bioactive agents.

22. The implantable medical device of claim 21 wherein said one or more bioactive agents is selected from the group consisting of anti-proliferatives, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides, transforming nucleic acids, cytostatic compounds, toxic compounds, chemotherapeutic agents, analgesics, antibiotics, protease inhibitors, statins, nucleic acids, polypeptides, growth factors, delivery vectors, liposomes, and combinations thereof.

23. A H₂S donating vascular stent comprising:
a stent; and
a polymer coating disposed upon said stent, wherein said polymer has the composition of Formula 5 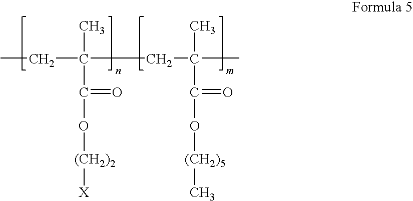
wherein X is a charged H₂S complex having the structure of Formula 2 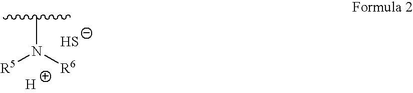
wherein R⁵ and R⁶ are each independently hydrogen or C₁ to C₂₅ straight chain alkyl; and
wherein n and m are each independently an integer of between 1 and 25,000.

24. The H₂S donating vascular stent of claim 23 further comprising a primer coating disposed on said stent.

25. The H₂S donating vascular stent of claim 24 wherein said primer coat is parylene.

26. The H₂S donating vascular stent of claim 23 further comprising a cap coat disposed on said stent.

27. The H₂S donating vascular stent of claim 26 wherein said cap coat is parylene.
